(19) United States
(12) Patent Application Publication
(10) Pub. No.: US 2003/0082158 A1
(43) Pub. Date: May 1, 2003

(54) Production of transduced hematopoietic progenitor cells

(76) Inventors: Geoffrey Symonds, Rose Bay (AU); John Murray, South Coogee (AU); Gregory Fanning, Bronte (AU); Janet MacPherson, Leichhardt (AU); Susan Pond, Lindfield (AU)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/192,058

(22) Filed: Jul. 10, 2002

Related U.S. Application Data
(60) Provisional application No. 60/304,283, filed on Jul. 10, 2001.
(60) Provisional application No. 60/343,392, filed on Oct. 22, 2001.

(51) Int. Cl. A61K 48/00; C12Q 1/68; C12N 15/85; C12N 5/08

(52) U.S. Cl. 424/93.21; 435/6; 435/455; 435/372

(57) ABSTRACT

The present invention relates to a method for introducing exogenous nucleic acid-containing hematopoietic progenitor cells into a patient.

This invention claims priority from U.S. Provisional Patent Application No. 60/304,283 filed Jul. 10, 2001 entitled “Method for Treating an HIV Infected Human” and U.S. Provisional Patent Application No. 60/343,392 filed Oct. 22, 2001 entitled “Production of Transduced Hematopoietic Progenitor Cells” the contents of which are incorporated by reference herein in their entirety.

### FIELD OF THE INVENTION

The present invention relates to gene therapy, particularly as applied to hematopoietic progenitor (HP) cells. More particularly, the present invention relates to the production of an enriched pool of transduced HP cells for delivery to a human subject to achieve a desired therapeutic effect and to methods of making and using the enriched pool of transduced HP cells.

### BACKGROUND TO THE INVENTION

For purposes of this invention, gene therapy refers to the deliberate introduction of recombinant DNA sequences into particular cell types for therapeutic benefit. Gene therapy may involve the introduction of a required gene or the use of other nucleic acid constructs to inactivate aberrantly expressed genes. Gene therapy may be aimed at a variety of diseases in which there is a genetic defect, for example.

A number of investigators have proposed and tested a variety of gene therapy approaches using novel anti-Human Immunodeficiency Virus agents in tissue culture. These approaches include intracellular expression of transdominant proteins (Smythe et al. 1994), intracellular antibodies (Marasco et al. 1998), antisense ribonucleic acid (RNA) (Sczakiel et al. 1991), viral decoys (Kohn et al. 1999), and catalytic ribozymes (Sarver et al. 1990; Sun et al. 1994, Sun et al 1996).

Ribozymes are small catalytic RNA moieties capable of cleaving specific RNA target molecules, including, for example HIV-1 and other strains of HIV. For example, ribozymes directed against HIV-1 can interfere with HIV-1 replication by interfering in several steps in the HIV-1 life cycle including (i) the production of genomic viral RNA in recently infected cells (prior to reverse transcription) and (ii) the production of viral RNA transcribed from the provirus before translation or prior to genomic RNA packaging (Sarver et al. 1990; Sun et al. 1994; Sun et al 1996; Sun et al. 1998). Generally, ribozymes are believed to be more effective than antisense-based therapies because ribozymes are catalytic molecules where a single catalytic ribozyme is capable of binding to and cleaving multiple RNA substrate molecules within a cell (Sarver et al. 1990; Sun et al, 1994; Sun et al. 1996).

The requirements for cleavage by a ribozyme are an accessible region of RNA and, in the case of the hammerhead ribozyme, the need for a GUX target motif (where G is guanosine and X is A, C or U ribonucleotides; in certain cases NUX may suffice (where N is any ribonucleotide and X is A, C or U ribonucleotides). Unlike some other anti-viral therapies, catalytic RNAs are unlikely to provoke an immune response. Unwanted immune responses expressing the catalytic RNAs could result in the elimination of cells that contain the exogenous gene.

A number of studies have demonstrated ribozyme cleavage activity in test tube reactions, and protective effects in tissue culture systems against laboratory and clinical isolates of HIV-1 (Sarver et al. 1990; Sun et al, 1994; Sun et al 1996; Sun et al. 1998; Wang et al. 1998). These studies used either hammerhead or hairpin ribozymes; for example, a hammerhead ribozyme, denoted as Rz2, directed against a highly conserved region of the tat gene, (FIG. 1). The tat gene is essential for HIV-1 replication; it encodes and produces the Tat protein that is a transcriptional activator of the integrated HIV provirus. The Rz2 ribozyme includes complementary hybridizing and target sequences that comprise nucleotides 5833-5849 (GGAGCCA GUA GAUCCUA, SEQ ID NO: 1) of reference strain HIV-HXB2 (Genbank accession number K03455) and similarly can target nucleotides 5865 to 5881 (GGAGCCA GUA GAUCCUA, SEQ ID NO: 1) of HIV IIIB (Genbank accession number X01762). In these studies the Rz2 ribozyme sequence 5′-TTA GGA TCC TGA TGA GTC CGT GAG GAC GAA ACT, GGC TC-3′, SEQ ID NO:2 was inserted as DNA into the 3′ untranslated region of the neo^{R} gene within the plasmid pLNL6, which contains the replication-incompetent retroviral vector LNL6 (Bender et al, 1987;Genbank accession number M63653 and see definition, infra) to generate a new virus, RRz2. The ribozyme sequence was expressed as a neo^{R}-ribozyme fusion transcript from the Moloney Murine Leukemia Virus (MoMLV) Long Terminal Repeat (LTR) in RRz2. Use of this virus successfully cleaved HIV infected cells in vitro.

The introduction of a therapeutic gene into CD34+ pluripotent hematopoietic progenitor cells ex vivo is an attractive possibility for the treatment of HIV-1 infection, since these progenitor cells may be readily separated from more mature hematopoietic cells (note that the CD34+ antigen is a membrane-bound 115 Kd molecule present on cells that are capable of giving rise to multilineage colony forming cells, but absent on more mature hematopoietic cells (Baum et al. 1992)) and are capable of relatively rapidly reconstituting lymphoid (CD4+ and CD8+ T-lymphocytes) and myeloid (monocyte/macrophages) hematopoiesis (Levinsky 1989; Schwartzberg et al. 1992). The hematopoietic progenitor (HP) cells differentiate and mature to give rise to cells of increasing maturity of the various lineages through intermediate progenitor cells. Key cells in terms of HIV/AIDS infection are the CD4+ T-lymphocytes and the monocyte/macrophages. Notwithstanding the time required for reconstitution, a single CD34+ HP cell is theoretically capable of reconstituting the entire hematopoietic system consisting of cells of varying stages of maturity within the various lineages. That said, from a practical standpoint, the optimal number of transduced HP cells that could efficiently repopulate the hematopoietic system with a gene modified cell population and could thereby impact on disease was not known.

Two Phase I clinical trials have been conducted using RRz2 by introducing the construct into either CD4+ (Cooper et al, 1999) or CD34+ (Amado et al, 1999) cells. In each of these trials, approximately half of each relevant cell population (CD4+ or CD34+) was transduced with LNL6 and the other approximately half transduced with RRz2, following which the cells were mixed and reinfused using methods that differed from the approach described herein. In the CD4+ trial, RRz2 containing lymphocytes were taken from HIV negative donors, transduced ex vivo and introduced into twin siblings who were genetically identical (Cooper et al, 1999).

In the CD34+ trial, the introduction of RRz2 into CD34+ cells ex vivo and infusion of these cells into the same patient was shown to be technically feasible and safe and resulted in ribozyme construct presence and expression in peripheral blood lymphoid and myeloid cells. The study's purpose was at least in part to render cells in an HIV infected individual at least partially protected from HIV-1 infection and HIV-1 intracellular replication. Indeed the studies demonstrated preferential survival of RRz2-containing lymphocytes over LNL6-containing lymphocytes in the CD34+ trial. Unlike the present invention, the Phase I HP trial was performed using a lower mean cell number being returned per patient and employed reduced transduction efficiencies than what is suggested in the present invention. Instead the Phase I trials were established to assess, the ability and safety of the ex vivo approach, and to determine the length of presence (persistence) of the hematopoietic cell progeny of these transduced cells in a patient. It is known that CD34+ cells have a large reconstitution and repopulation potential, and there is evidence that CD34+ cells are not directly infected by HIV. In part, the present invention relates to the identification of a therapeutically relevant level of transduced CD34+ cells to provide an ongoing source of protected cells within a patient thereby impacting disease progression.

To impact on diseases progression, not only for HIV infection, but for other diseases involving cells of the hematopoietic system, there is a need to define and possibly maximise the numbers of transduced HP cells in order to produce sufficient numbers of genetically modified precursors of lymphoid and myeloid cells that will produce genetically modified mature lymphoid and myeloid cells within a reasonable amount of time.

It is held by the present inventors that in order to achieve a benefit to the patient with genetically modified HP cells, that have been transduced ex vivo, it is necessary that the recipient patient receive a sufficient number of transduced HP cells to produce a chimeric hematopoictic system that will yield enough ribozyme-containing mature lymphoid (CD4+ and CD8+ T-lymphocytes) and myeloid (monocyte/macrophages) cells to impact on viral infection and/or disease progression. This is also true for any disease, viral or not where genetically modified HP cells or more mature progenitor cells of the hematopoietic lineage are needed. In these diseases the HP cells are identically isolated, processed, and transduced to give rise to gene-containing progeny that can be reintroduced into a patient and can then become established, or engrafted in the bone marrow of that patient. The present invention is therefore directed at defining, obtaining and preparing the required therapeutic dose of an enriched pool of HP cells transduced with a therapeutic gene for delivery to the patient, this enriched pool being derived from a population of HP cells, including CD34+ cells. The rationale is that these transduced HP cells will give rise to mature lymphoid and myeloid cells containing the therapeutic gene.

### BRIEF DESCRIPTION OF THE ACCOMPANYING FIGURES

FIG. 1 provides an illustration of the location of the ribozyme target site within the HIV-1 genome. FIG. 1A provides a schematic diagram of the HIV-1 genome showing location of replicative, regulatory and accessory genes; FIG. 1B provides a preferred the ribozyme sequence together with its complementary target and hybridizing sequence within the tat gene. The target GUA cleavage site is circled; and FIG. 1C provides the location of a GUA target sequence in the genes encoding Tat and Vpr proteins.

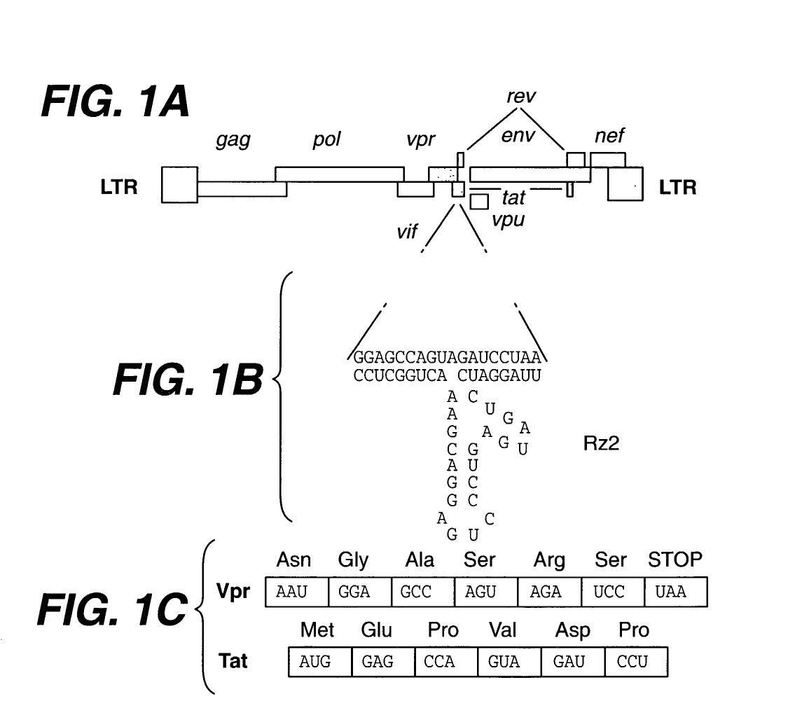

FIG. 2 is a flow chart of the exemplary steps of the present invention. Preferably, the steps in this example are carried out in a sequential manner as shown.

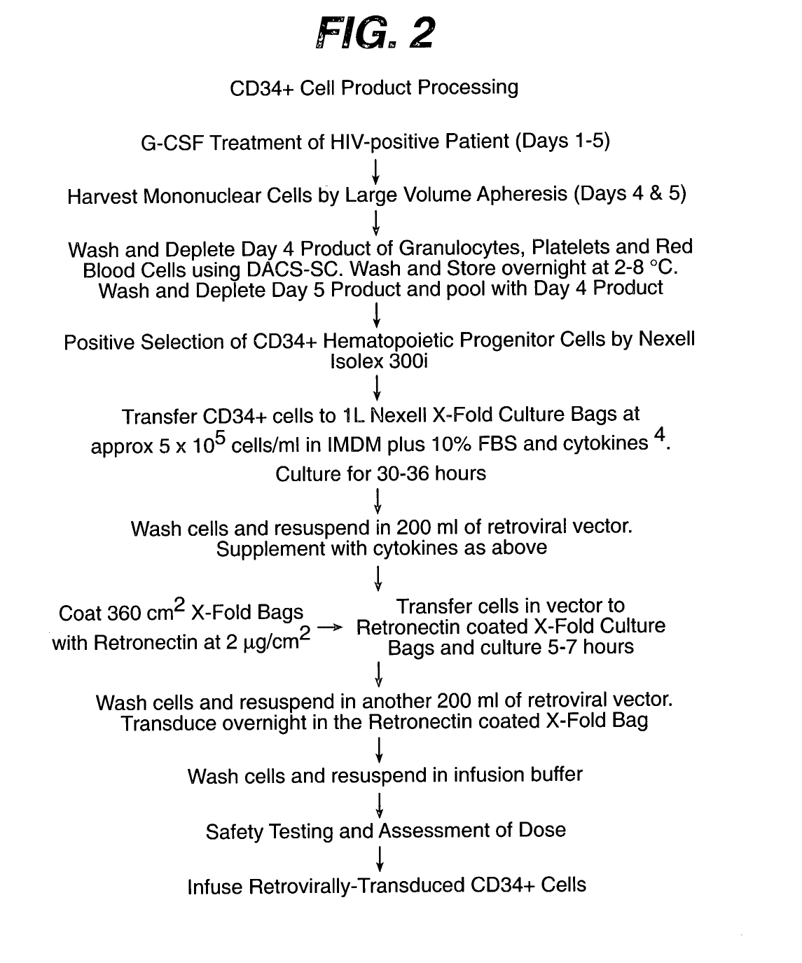

FIG. 3 illustrates the principal of real-time quantitative PCR, in this case, DzyNA PCR, for the determination of the percentage of gene-containing and gene-expressing cells. FIG. 3A is a schematic of the DzyNA PCR detection method and FIG. 3B shows the means of quantitation.

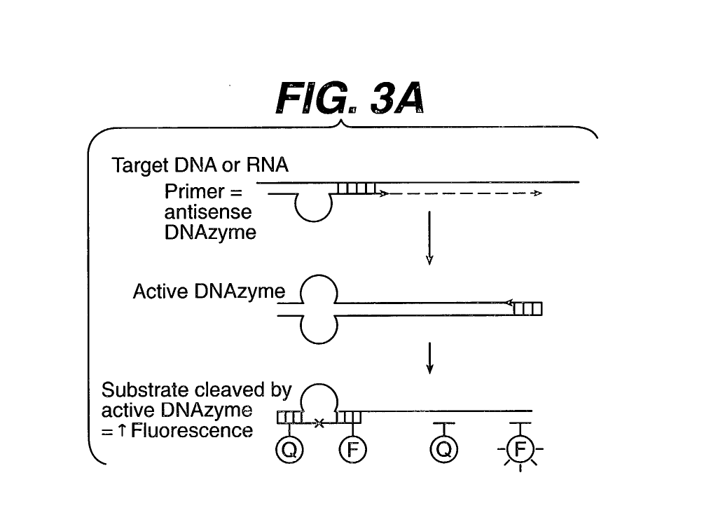

FIG. 4 illustrates the mathematical model for CD4+ T lymphocyte production from CD34+ HP cells. The parameters that have been considered are the rate at which T lymphocyte precursors leave the bone marrow, pass through the selection mechanisms within the thymus, and are exported as naïve cells (N) into the peripheral blood. This requires estimation of thymic export for individuals at different ages, because it is known that the thymus involutes with age and its rate of CD4+ T lymphocyte export decays accordingly (Sempowski et al, 2000). Once established as naïve cells in the periphery, the survival and expansion of gene-containing T lymphocytes is dependent on the natural homeostatic mechanisms. The natural mechanisms regulating T cell numbers are depicted in the Figure, and they comprise processes (1)-(4) which are the following aspects of CD4+ T lymphocyte development: export of new naïve cells from the thymus (1); activation of naïve (2), and memory cells to generate activated cells, some of which revert to a memory phenotype (3); and reversion of memory cells to a naïve phenotype (4).

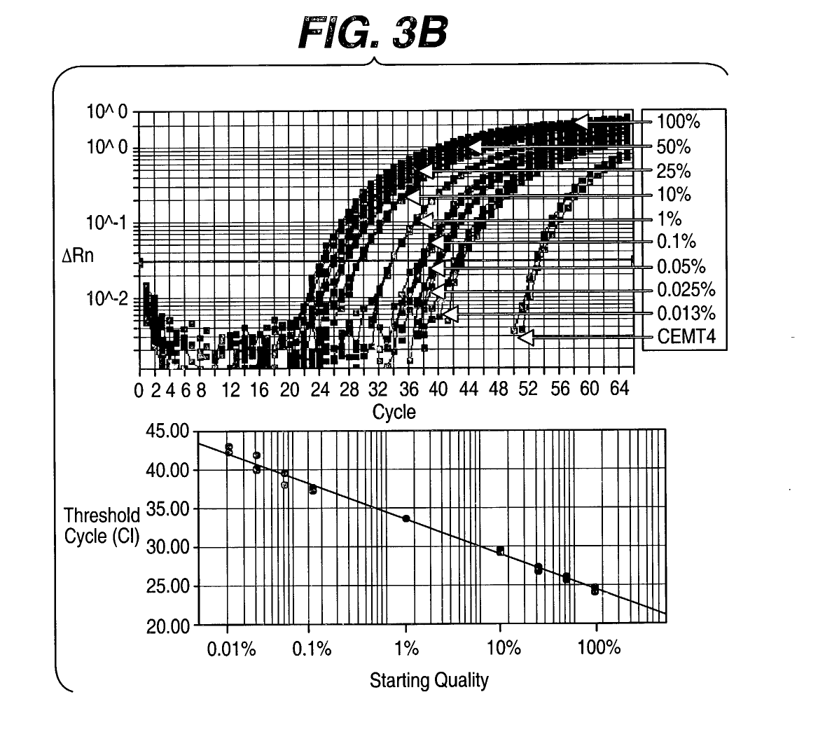

FIG. 5 illustrates the mathematical model for macrophage production from CD34+ HP cells in the bone marrow.

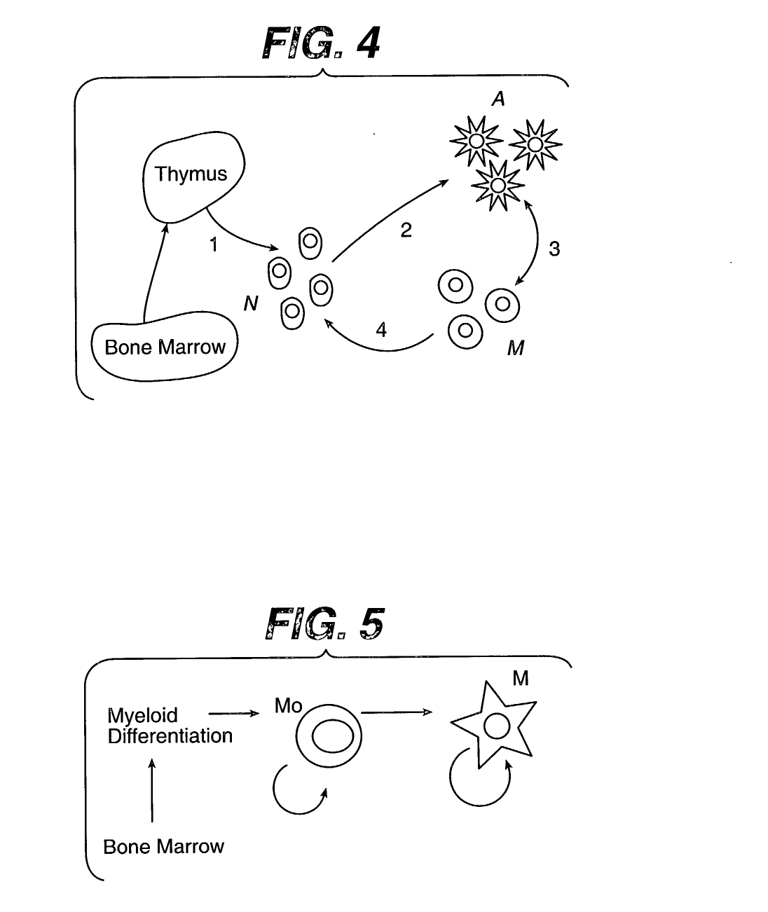

FIG. 6 illustrates the mathematical model for CD4+ T lymphocyte production from CD34+ HP cells in the presence of HIV-1 infection. The parameters that have been considered are the rate at which RRz2 (or other anti-HIV gene)-containing T lymphocyte precursors leave the bone marrow, pass through the selection mechanisms within the thymus, and are exported as naïve cells (N) into the peripheral blood. This requires estimation of thymic export for individuals at different ages, because it is known that the thymus involutes with age and its rate of CD4+ T lymphocyte export decays accordingly (Sempowski et al, 2000). Once established as naïve cells in the periphery, the survival and expansion of RRz2-containing T lymphocytes is dependent on the natural homeostatic mechanisms and a potential selection advantage over Rz2-CD4+ T lymphocytes when HIV alters these mechanisms. The natural mechanisms regulating T cell numbers are depicted in the left-hand side of the Figure, and they comprise processes (1)-(4) which are the aspects of CD4+ T lymphocyte development detailed in the text relating to FIG. 3.

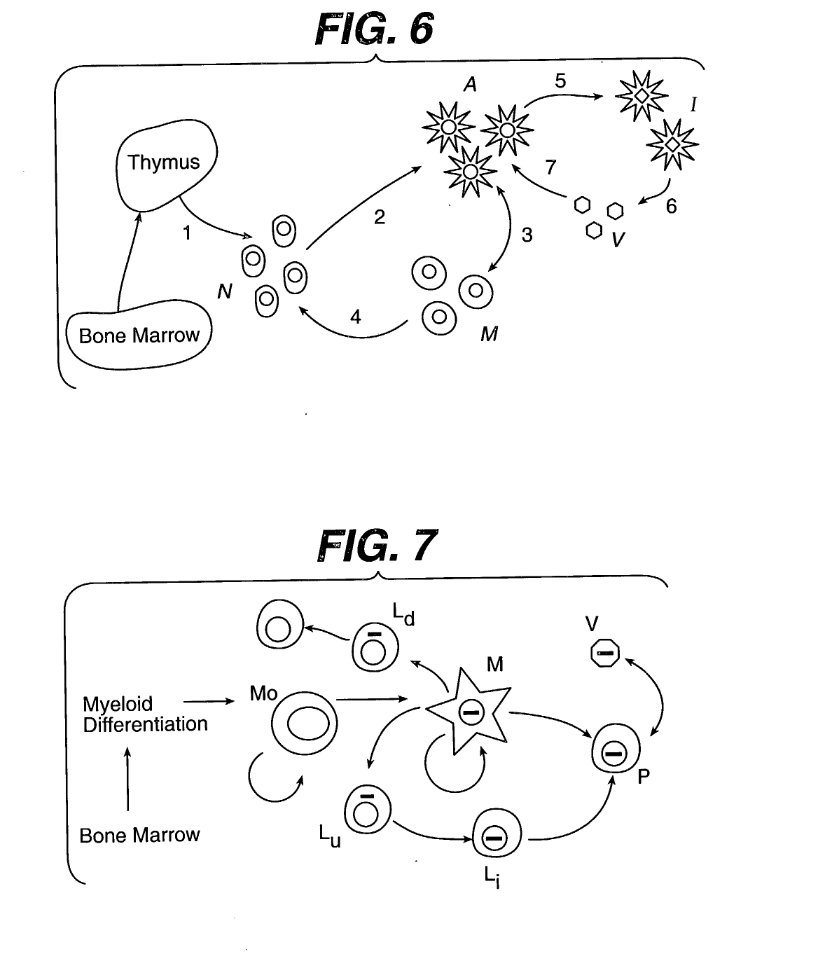

In addition to thymic export, RRz2 containing CD4+ T lymphocytes increase in number through activation by antigen and expansion into the memory T Lymphocytes. The present model of this invention incorporates estimates of the degree to which this memory T Lymphocyte expansion occurs. With infection by HIV, the components in the right hand side of the Figure (processes (5) through (7)) come into play. Activated cells are infected by virus (5,7); these in turn produce new virus (6), which completes the cycle of infection. These mechanisms are included in the model. Additionally the model allows for alteration in some of the natural processes such as increased activation of naïve and memory cells (2,3), due to the presence of HIV.

FIG. 7 illustrates the mathematical model for macrophage production from CD34+ HP cells in the presence of HIV-1 infection. The model is based on the belief that infected monocytes and macrophages plays an important role in maintaining infection during administration of anti-retroviral therapy and that these cells significantly contribute to ongoing infection when anti-retroviral therapy is not used. A model that assesses the contributions of this infected component has been developed here and incorporates some of the hypotheses of Zack et al, 1990 and Murray et al, 2001. The present model examines HIV-1 RNA and HIV-1 DNA dynamics in both untreated and treated seroconverters. It takes into account the labile nature of unintegrated HIV-1 DNA and includes latently infected CD4+ T lymphocytes and infected macrophages. The model incorporates infection in an unintegrated form, both defective L_{d}, and competent Lᵤ, through interaction with long-lived infected macrophages M. Latently infected cells with integrated HIV-1 DNA Lᵢ, arise from competent unintegrated HIV-1 DNA Lᵤ, as the HIV-1 DNA molecule is integrated. Productively infected cells P, arise from activated cells infected by free virus V, latently infected cells with integrated HIV-1 DNA being activated, and through cells activated during the process of interaction with infected macrophages. Macrophages are infected through contact with infected macrophages.

### SUMMARY OF THE INVENTION

In one aspect of the present invention, the invention relates, through mathematical modeling, to the identification of a minimum threshold number of genetically engineered HP cells, which, following transduction and reinfusion, are useful for ensuring that a significant proportion, if not the entire hematopoietic system, is repopulated with genetically modified cells of the various blood cell lineages, such that the gene modified cells have a therapeutic effect.

Further, the invention relates to a method for achieving this minimum threshold number of therapeutic gene-containing hematopoietic progenitor (HP) cells. The method comprises, in addition to cell washing steps: mobilization of the HP cells from the bone marrow to the peripheral blood compartment of the patient; apheresis of the blood to obtain the mononuclear cell fraction; purification of the HP cell population by using CD34 antigen or hematopoietic-depletion antigens; transfer of the HP cells to tissue culture; cytokine/growth factor activation and culture; retroviral transduction; subsequent cell culture; harvest; and re-infusion to the patient. The invention further uses the model to generate a quantitative measurement of the transduced HP cells and a quantitative measurement of the gene-containing progeny cells within an individual. The latter provides a means to monitor the degree of gene-modified chimerism of the hematopoietic system, as an indicator of potential therapeutic benefit.

### DETAILED DESCRIPTION OF THE INVENTION

The term “Hematopoietic Progenitor (HP) cells” refers to hematopoietic cells that are pluripotential and continuously give rise in vivo to all of the various lineages of the hematopoietic system.

The term “CD34+ cells” refers to cells which have the CD34+ antigen on their surface. They are a subset of hematopoietic progenitor cells.

The phrase “purity of CD34+ cells” refers to the percentage of cells in any population that is positive for CD34 antigen.

The term “exogenous nucleic acid product” refers to an expressible nucleic acid fragment that is introduced into a cell, preferably a fragment when introduced into the cell has a therapeutic effect, preferably an anti-viral effect. Also preferably the fragment is non-native to the cell. This product can include, but is not limited to a gene encoding a protein, including an antibody, an antisense molecule, a ribozyme, or other product that can be generated through transcription or transcription and translation within the cellular milieu.

The term “LNL6” refers to a murine retroviral vector, derived from Moloney Murine Leukemia Virus, that has the replicative genes deleted and the neomycin phosphotransferase (neo^{r}) gene inserted (Bender et al, 1987). The vector is based on the retroviral plasmid, pLNL6, which contains the replication-incompetent retroviral vector LNL6 (Genbank accession number M63653).

The term “Rz2” refers to an anti-HIV hammerhead ribozyme targeted to a highly conserved region of the tat gene). The Rz2 ribozyme sequence in the DNA form is 5′-TTA GGA TCC TGA TGA GTC CGT GAG GAC GAA ACT GGC TC-3′, SEQ ID NO:3 and in the RNA form is 5′-UUA GGA UCC UGA UGA GUC CGU GAG GAC GAA ACU GGC UC-3′, SEQ ID NO:4.

The term “RRz2” refers to a retroviral vector consisting of LNL6 with Rz2 inserted into the 3′ untranslated region of neo^{r}.

The term “DzyNA” refers to a method for real-time quantitative PCR detection and quantification of DNA or RNA such as that described in U.S. Pat. No. 6,140,055 and U.S. Pat. No. 6,201,113.

The term “transduction” refers to the introduction of a gene into a cell and the consequent expression of that gene in that cell.

In a first aspect, the present invention provides for the determination of dose and method of preparing cells containing an exogenous therapeutic gene(s) for delivery to a subject. To determine the effect of giving an increased HP cell dose to a patient, unique mathematical simulations were generated. These simulations were used to predict whether or not the gene-transduced HP cell approach could produce a clinically relevant effect on mature T lymphoid and monocyte/macrophage progeny cell populations.

Mathematical modeling was used to address the dynamics of two cell populations; i) CD4+ T lymphocytes, and ii) monocytes together with their progeny tissue macrophages. Modeling for each cell type was performed separately as the cells are characterized by different cell growth, maturation and death parameters. For example, the amount of viral reduction due to the Rz2-containing populations is determined, and in the case of CD4+ T lymphocytes, the extent to which the CD4+ T lymphocyte population is maintained/increased in the presence of HIV is evaluated.

Mathematical simulation was based at least in part on published differential equations (Murray et al. 1998, Haase, 1996) that describe: (i) T lymphocyte cell production over time as a function of age of the individual and mass of the thymus; (ii) Naïve T lymphocyte activation and proliferation in response to antigen; and (iii) Monocyte/macrophage production.

These simulations indicated that increasing the dose of HP cells to a minimum threshold level could give rise to increasing numbers of gene-containing CD4+ T lymphoctyes and monocyte/macrophages. Results indicated that when the percentages of transduced CD34+ cells exceeded 10% of the resident HP cells and more preferably exceeded 20% of the resident HP cells this would impact on viral load and CD4+ cell counts. Moreover, it was only by separately modeling in both lymphocytes and macrophages that the model predicted the conditions under which an antiviral effect in the macrophages would be seen. An antiviral effect in macrophages is important for diminishing the reservoir of HIV virus within a subject.

In a second aspect, the present invention provides a method for production and delivery of this same percentage of cells containing a therapeutic gene. This method comprises:

(a) obtaining from a subject a cell population comprising CD34+ HP cells;

(b) concentrating the HP cells to provide a cell population comprising at least 40% HP cells; and

(c) introducing a vector comprising a gene into the HP cell population, wherein the gene is capable of being expressed in said HP cells and wherein the cells are further cultured in vitro; and

(d) determining the number of such gene-containing HP cells and non gene-containing HP cells such that upon delivery to the same or another subject, the same or another subject receives a dose of at least 0.52×10⁶ gene containing HP cells per kg body weight of the subject in a total cell population of 1.63×10⁶ HP cells per kg body weight.

Preferably the number of gene-containing HP cells is such that one is able to observe at least 10% gene-containing HP cells in the bone marrow of the subject at between about 1-3 months following the method. Modeling predicts that if that amount of gene-containing HP cells is present in the bone marrow a therapeutic effect, such as, for example, an antiviral effect will be observed. More preferably, the gene-containing CD34+ HP cells produce gene-containing progeny lymphoid and myeloid cells that can be detected in the individual's body for at least 1 year following the introducing step. Still more preferably the chimeric hematopoietic system produced as the result of this method will include at least 0.01%, 0.1%, 1.0%, 10% and more preferably 20%, and still more preferably 50% gene-containing cells in any of the peripheral blood cell types within 4 years following the introducing step. In addition, the chimeric hematopoietic system produced as the result of this method will include at least 0.01%, 0.1%, 1.0%, 10% and more preferably 20%, and still more preferably 50% gene-containing cells in a bone marrow sample obtained within 4 years following the introducing step.

Thus this invention also relates to a method for predicting whether or not a reduction in viral load in a subject is likely to be observed comprising the above method steps and wherein following engraftment (i.e., the time at which the cells establish themselves in the bone marrow) there are at least 10% gene-containing HP cells in the bone marrow. Further, in another preferred embodiment of this invention, if the number of gene-containing and/or non gene-containing HP cells is less than the preferred number for introduction into the subject, then the cells are frozen and one or more additional mobilization and aphereses are conducted until the pooled HP cell numbers (gene and non gene-containing) is at least the amount as provided in step (d) above.

Preferably, the resultant pool of cells comprises sufficient gene(s)- containing CD34+ HP cells such that, upon delivery to said subject, the subject receives a dose of at least 5×10⁶, more preferably 1×10⁷, more preferably in excess of 2×10⁷ CD34+ HP cells and even more preferably in excess of 4×10⁷ or 5×10⁷ CD34+ HP cells containing the gene(s) per kg body weight of the subject and still more preferably 8×10⁷ or 10×10⁷ CD34+ HP cells containing the gene(s) per kg body weight of the subject

Preferably, the resultant pool of cells is such that, upon delivery to said subject, the subject receives a total number of cells (i.e. the HP cells containing the therapeutic gene(s) and all other cells present in the resultant pool of cells) of at least 1×10⁷/kg body weight of the subject up to 4×10⁷ cells/kg or more preferably up to 10×10⁷ cells per kg or more.

The population of cells “harvested” from the subject may be obtained by any number of methods well known in the art. For instance, the patient may be treated so as to mobilize HP cells from bone marrow into the peripheral blood, for example by administering a suitable amount of a cytokine including, but not limited to, pegylated Granulocyte—Colony Stimulating Factor, pegG-CSF, Granulocyte Macrophage Colony Stimulating Factor (GM-CSF) and, more preferably G-CSF, followed by apheresis filtration. Alternatively, HP cells may be aspirated from bone marrow or cord blood in accordance with well-known techniques.

Treatment of the harvested population of cells preferably includes one or more washing steps (e.g. using centrifugation or automated cell washers) and/or de-bulking steps (i.e. to remove excess red blood cells, granulocytes, platelets, T-lymphocytes and). Preferably, the debulking step is performed using a device such as the Dendreon DACS System (Charter Medical, Winston Salem, N.C.) and, preferably further comprises a HP cell selection step. HP cell selection may be achieved by immune affinity or flow cytometry techniques. Preferably, the HP cell selection step selects CD34+ cells or in another embodiment may involve antigen depletion of mature/committed hematopoietic cells, thereby enriching the cell population for HP cells. The HP cell selection step can be performed using a variety of selection devices such as, but not limited to, the Nexell/Baxter Isolex 3001 (Irvine, Calif.), the Miltenyi CliniMACS,(Miltenyi; Biotech GMBH, Bergisch Gladbach, Germany), Stem Cell Technologies (Vancouver, BC, Canada) StemSep Device.

The treatment of the harvested population of cells may also involve a cell culturing step to increase cell numbers and especially to increase the number of selected HP cells. Cell culturing is also required to introduce the therapeutic gene(s) into the cells and cell culturing may be used after introduction of the therapeutic gene(s) to facilitate gene integration and expression of the gene construct and to preferably expand the number of such gene(s)-containing HP cells.

The initial treatment steps (mobilization, apheresis, HP selection) results in the obtaining of, and enriching for, HP cells. The definition of the percentage of HP cells requires a measurable aspect of these cells such as CD34 antigen positivity. It is to be understood that the treated pool of cells ex vivo preferably comprises at least 20%, more preferably at least 40%, more preferably still at least 60% and most preferably at least 80% HP cells.

Introduction of the therapeutic gene(s) or nucleic acid sequence(s) into at least a portion of the HP cells may be achieved with any of a variety of methods well known to the art or other methods. In a preferred embodiment, the introducing step employs, transduction using retroviral vectors or other viral or non-viral (DNA or RNA) vectors carrying the therapeutic gene(s) or nucleic acid sequence(s). Where transduction is used, a transduction-facilitating agent (e.g. for retroviral vectors, the CH296 fragment of fibronectin known as RetroNectin or other agents such as polybrene or protamine sulphate) is preferably used. The HP cells containing the therapeutic gene(s) or nucleic acid sequence(s), and cells derived therefrom (i.e. from subsequent lymphoid and mycloid hematopoiesis), contain and are preferably capable of expressing the therapeutic gene(s) or nucleic acid sequence(s) where the therapeutic gene is intended for cell expression.

The therapeutic nucleic acid introduced into the HP cells can encode a product such as, but not limited to, proteins (e.g. transdominant proteins and intracellular antibodies), antisense RNA, aptamers, interfering RNA and catalytic ribozymes in the case of HIV/AIDS and these or other genes such as tumor suppressor genes in other diseases.

The cells may be delivered to the subject in accordance with routine methods such as cell infusion. The cells may be delivered together with a pharmacologically-acceptable carrier (such as, for example. 5% Human Serum Albumin) with pharmaceutically aceptable buffers, salts, and the like. The subject may or may not be first (ie before re-infusion of the cells) subjected to myeloablation of the bone marrow (defined as or other hematopoietic conditioning regimens. However, in a preferred method for engrafting a transduced HP cell population of the present invention and indeed, a substantial benefit of the present invention, the subject does not receive myeloablation therapy (i.e., complete or near complete destruction of the bone marrow) or other hematopoietic conditioning regimes. The benefit of these methods for generating a transduced and engrafted HP cell population without myeloablation is that it myeloablation procedures, including, but not limited to chemotherapeutic or radiation treatments, are toxic, energy draining and debilitating to those subjects receiving the procedures.

The methods of this invention may be combined with other therapies, including for example, other anti-viral therapies. Other antiviral therapies include for example, the use of RNA decoys, intracellular antibodies, interfering RNA (Sharp, P.A. (2001), RNA interference-2001 in Genes & Dev 15:485-490), and the like. For example, where the method is directed to the use of an anti-HIV therapy, such as for example a ribozyme-type therapy, other anti-viral or particularly anti-HIV therapies may be used. Where a ribozyme-type therapy is used, more than one catalytic ribozyme may be delivered to a cell or different ribozymes can be delivered to different cells in the sample originally removed from the patient. Similarly, the method can be combined with other gene therapies not requiring HP cell transduction, such as standard chemotherapies and protein therapies known in the art. One example with respect to the treatment of HIV-infected patients is the combination of the method of this invention with standard medicaments for HIV, particularly where HIV-resistance is detected or where HIV-infection in a subject has proved refractory to other anti-HIV treatments.

While this invention is contemplated as a general method for introducing exogenous gene containing cells of the hematopoietic system, the invention is directed, by way of example only to anti-viral gene therapy for HIV. In this method the harvested cells from an HIV positive subject are enriched for HP cells and the therapeutic gene(s) encodes an anti-HIV product(s).

Thus, in a third aspect, the present invention provides for defining the dose and preparing HP cells, preferably CD34+ cells, which contain a gene(s) encoding an anti-HIV product(s) for delivery to an HIV positive subject in order to consistently achieve an antiviral therapeutic effect. While ribozymes are contemplated as a preferred embodiment of this invention, other gene-encoding anti-viral products can be used such as, but not limited to antisense therapy, interfering RNA, and the like

As indicated above, the mathematical simulation for this determination is based on published differential equations (Murray et al. 1998, Haase, 1996) and the mathematical simulation used in this invention as applied to HIV infection takes into account:

i) T lymphocyte cell production over time as a function of age of the individual and mass of the thymus;

ii) Naïve T lymphocyte activation and proliferation in response to antigen;

iii) CD4+ cell decline over the course of HIV infection; and

iv) production of monocyte/macrophages.

The determination of the effect of increasing CD34+ dose was explored by using mathematical simulations which provided a theoretical method to assess whether or not the RRz2-transduced CD34+ cell approach could produce a clinically relevant effect on CD4+ cell count and viral load in HIV patients. These simulations predict that increasing the dose of CD34+ cells would give rise to RRz2-contining CD4+ T lymphoctyes and monocyte/macrophages which would impact on CD4+ T lymphocyte counts and HIV viral load. Based on these simulations we have elaborated a method to define and maximize the number of transduced HP cells introduced. The dose of transduced CD34+ cells is increased by a factor of at least 2-10 over the highest dose used in previous Phase I trials. This dose is attainable by the methodology described.

The method of delivery of the anti-HIV product(s) therefore comprises:

(i) obtaining from said subject a population of viable cells including HP cells, preferably CD34+ cells;

(ii) treating and/or culturing said population of cells to provide a pool of cells comprising at least 20% CD34+ cells; and

(iii) introducing at least one therapeutic gene into a population of the CD34+ cells within said pool of cells such that said therapeutic gene(s) is/are capable of being expressed in said CD34+ cells; wherein a resultant pool of cells is prepared which comprises CD34+ cells containing the therapeutic gene(s) such that, upon delivery to said subject, the subject receives a dose of at least 0.52×10⁶ HP cells containing the therapeutic gene(s)/kg body weight.

Preferably, the resultant pool of viable cells is prepared which comprises therapeutic gene(s) containing CD34+ HP cells such that, upon delivery to said subject, the subject receives a dose of at least 5×10⁶, more preferably in excess of 2×10⁷, and even more preferably in excess of 5×10⁷ HP cells containing the therapeutic gene(s)/kg body weight.

Preferably, the resultant pool of cells is such that, upon delivery to a patient, the patient receives a total number of cells (i.e. the HP cells containing the therapeutic gene(s) and all other cells present in the resultant pool of cells) of at least 1×10⁷/kg body weight up to 4×10⁷ cells/kg or more preferably up to 10 ×10⁷/kg or more).

The harvesting of the population of cells and subsequent treatment thereof, may be carried out as described above in respect to the first aspect of the invention. Preferably, the treatment involves a first step of washing the population of cells (e.g. using a standard cell washer), optionally followed by a step of de-bulking (e.g. using a standard apparatus for removal of excess red blood cells, granulocytes, platelets, T-lymphocytes) to produce a population of cells enriched for HP cells, a second step of washing (e.g. using an automated cell washer), and culturing the HP enriched population of cells.

The initial treatment steps (mobilization, apheresis, HP selection) results in an enrichment of the proportion of HP cells. The definition of the percentage of HP cells requires a measurable aspect of these cells such as CD34 antigen positivity. It is to be understood that the treated pool of cells preferably comprises at least 20%, more preferably 40%, still more preferably at least 60% and most preferably at least 80%, HP cells.

Introduction of the gene(s) into at least a portion of the CD34+ cells is preferably achieved through transduction of these cells with a retroviral vector carrying the therapeutic gene(s), in the presence of a transduction-facilitating agent (e.g. RetroNectin). However, those of ordinary skill in the art of gene therapy will recognize that other published methods for introducing a gene into a cell can produce equivalent results. The gene(s) may encode any anti-HIV product, but preferably encodes an anti-HIV catalytic ribozymes. Particularly preferred anti-HIV-1 catalytic ribozymes are those which cleave HIV RNA within the tat gene and, particularly, within the highly conserved region of that gene (i.e. nucleotides 5833-5849 (GGAGCCA GUA GAUCCUA, SEQ ID NO:3) of reference strain HIV-HXB2 (Genbank accession number K03455) and nucleotides 5865 to 5882 (GGAGCCA GUA GAUCCUA) of the HIV-IIIB strain (Genbank Accession number X01 762))

In a preferred embodiment, the subject does not require myeloablation of the bone marrow or other marrow conditioning regimen, and the step of delivering the cells results in the subject receiving a dose of at least 1.63×10⁶ CD34+ cells/kg body weight and of this population at least 0.52×10⁶ CD34+ cells containing the therapeutic gene(s)/kg body weight.

The present invention further relates to methods to monitor for the presence and expression of the gene construct. We have developed a quantitative real time PCR methodology for this detection. This type of quantitative real time PCR methodology, termed DzyNA-PCR is disclosed and described by Todd et al. 2000 and in U.S. Pat. Nos. 6,140,055 and 6,201,113 where a strategy is provided for the detection of specific genetic sequences associated with disease or the presence of foreign agents. The method provides a system that allows homogeneous nucleic acid amplification coupled with real-time fluorescent detection in a single closed vessel. The strategy involves in vitro amplification of genetic sequences using a DzyNA primer which harbors the complementary (antisense) sequence of a 10:23 DNAzyme (Santoro et al. 1997). During amplification, amplicons are produced which contain active (sense) copies of DNAzymes that cleave a reporter substrate included in the reaction mix. The accumulation of amplicons during PCR is monitored by changes in fluorescence produced by separation of fluoro/quencher dye molecules incorporated into opposite sides of a DNAzyme cleavage site within the reporter substrate. Cleavage of this reporter substrate indicates successful amplification of the target nucleic acid sequence. Real-time measurements can be performed on the ABI PRISM® 7700 Sequence Detection System (Applied Biosystems) or other thermocyclers that have the capacity to monitor fluorescence in real time.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention before the priority date of each claim of this application.

The invention will hereinafter be described with reference to the following non-limiting examples and accompanying figures. All publications cited in this document are incorporated by reference herein.

### EXAMPLES

### Example 1

### HP Cell Harvesting Transduction and Re-Infusion.

In a preferred method, the invention comprises the following steps:

1. HP Cell Mobilization from the individual's bone marrow into the peripheral blood;

2. Apheresis of the peripheral blood of the individual to obtain the mobilized-HP cells;

3. Washing Step #11 of the unpurified peripheral blood mononuclear cells by using a cell washer in preparation for potential de-bulking;

4. De-bulking Step; to remove excess red cells, granulocytes, platelets, and T-lymphocytes;

5. Washing Step #2: of the enriched HP cells using a cell washer

6. CD34+ Cell Selection or depletion of antigen positive cells from the HP cell population;

7. Washing Step #3; of the purified HP cells using a cell washer;

8. Cell Culture by placing the purified HP cells into culture with cytokines/growth factors;

9. Transduction Procedure of the HP cells by using a retroviral vector containing the gene construct in the presence of a transduction-facilitating agent; introducing the viral vector;

10. Harvest Cell Product and wash the HP cells, including the transduced HP cells;

11. Preparation of Infusion Product; place the HP cells into an infusion bag and perform product safety release testing; and

12. Infusion of Patient cells back into the same individual.

These steps are described as follows with examples and other modifications to the invention provided below:

Step 1-HP Cell Mobilization.

The first step of this procedure uses an agent to mobilize HP cells from the bone marrow into the peripheral blood. An example here is the use of a cytokine selected from the preferred group comprising pegylated Granulocyte—Colony Stimulating Factor (pegG-CSF), Granulocyte Macrophage, GM-CSF and, most preferably G-CSF followed by apheresis filtration. Alternatively, HP cells may be aspirated from bone marrow or cord blood in accordance with well-known techniques.

Granulocyte Colony Stimulating Factor (G-CSF), (Amgen, Thousand Oaks, Calif., Neupogen™) is administered to the patient subcutaneously, at least at 10 μg/kg/day and preferably at about 30 μg/kg/day, once daily, for up to five consecutive days. Complete Blood Counts (CBCs), differential and platelet count are performed daily during G-CSF administration to assess the extent of the leucocytosis. A blood sample to determine CD34+ cell counts is preferably drawn on day 3 of G-CSF administration to ensure that the peripheral blood CD34+ count is greater than 20 cells/mm³ prior to the start of apheresis. Failure to attain this CD34+ cell number does not however prevent apheresis which generally occurs on days 4 and 5 of G-CSF administration.

Step 2-Apheresis (Example 1: Preferably on Days 4&5).

Apheresis is a method of “blood filtration” to obtain the mononuclear cell fraction of the peripheral blood. Here, a Cobe Spectra (Gambro BCT, Lakewood, Colo.), Haemonetics (Haemonetics Corporation, Braintree, Mass.) or Amicus (Baxter Fenwal, Deerfield, Ill.) machines are preferably used on at least two separate occasions, (preferably on days 4 and 5 following mobilization, where day 1 is the first day of induced mobilization), though in other examples apheresis can be done on earlier or later days by determining the day at which the peripheral blood CD34+ count is greater than: 5 cells/mm³ or more preferably 10 cells/mm³ and most preferably 20 cells/mm³. In a preferred embodiment, this apheresis yields cellular product from about 5 Liters (L) of blood flow through, preferably this will be 5-10 L, but more preferably 10-20 L, and more preferably still 20 L or greater. Product from each apheresis is either treated separately or, in a preferred embodiment, pooled after the second apheresis. Total cell counts, and absolute CD34+ cell numbers are recorded. Use of Steps 1 & 2 will produce up to or greater than 5×10⁶, preferably greater than 2×10⁷, more preferably greater than 4×10⁷ HP (as measured by CD34 positivity) cells/kg.

Step 3-Washing Step #1 (Example 1: Preferably on Days 4&5).

The pooled cells are washed. This is done by cell centrifugation (1,500 rpm or 300 g, 15 minutes or similar) or more preferably using an automated cell washer, in one example this cell washing is done by using a Nexell CytoMate washer (Nexell Therapeutics, Irvine, Calif.) using a program that washes cells from bag 4 through spinning membrane to wash bag 1 with the following parameters (Residual Fold Reduction=1; Maximum End Weight=190 mL; Source Bag Rinse=50 mL or similar). This is followed by transfer of cells from wash bag 1 to bag 3 end product with the following parameters (Tubing Rinse Volume=90 mL; Maximum Pump Rate=50 mL per minute; or similar parameters).

Step 4-De-Bulking Step (Example 1; Preferably on Days 4&5).

In one embodiment, the cells from the apheresis procedure(s) are “de-bulked” on each apheresis day—using a system like a Charter Medical DACS-SC™ system (Charter Medical, Winston-Salem, N.C.). In the embodiment where product is stored overnight from the first (or consequent) day(s) for subsequent pooling with final day product, the two or more apheresis products are de-bulked on the day of collection and the first product stored until the second product has been de-bulked. De-bulking involves loading the washed apheresis cell product onto the BDS60 solution within the DACS-SC™ device and centrifuging at 850 g for 30 minutes at 20-25 degrees Celsius without a brake. The product is recovered by inversion and collection into a transfer pack under sterile conditions.

Step 5-Washing Step #2 (Example 1; Day 4).

On the day or days of collection that will be stored before pooling, the resultant product is subjected to a wash step using Dulbecco's phosphate buffered saline (DPBS) supplemented with 0.5% human serum albumin. This is done using a Nexell CytoMate washer using a program that washes cells from bag 4 through spinning membrane to wash bag 1 with the following parameters (Residual Fold Reduction=1,000; Maximum End Weight=20 mL; Source Bag Rinse=50 mL; or similar parameters). This is followed by transfer of cells in 100% autologous plasma from wash bag 1 to bag 3 end product with the following parameters (Tubing Rinse Volume=195 mL; Maximum Pump Rate=50 mL per minute; or similar parameters). The fluid path is then washed with an additional 50 mL of the DPBS plus 0.5% human serum albumin. The cell product is then stored overnight at a cell density of not greater than 200×10⁶ cell per mL at 2-8 degrees Celsius. The product that will not be stored overnight is not subjected to a separate wash step as this occurs during the CD34+ selection step (Step 6 below).

Step 6-CD34+ Cell Selection (Example 1; Day 5).

The cells are taken, counted, pooled (in the embodiment where there are two or more products) and placed into the LifeCell bag (Nexell Therapeutics, Irvine, Calif.) from the Isolex 300i (Nexell Therapeutics, Irvine, Calif.) disposable set. (If there are more than two products all will be pooled at the latest time point). CD34+ cells are selected from the post-washing product by using the Isolex 300 i, Miltenyi or a lineage depletion strategy of cells expressing markers (e.g. CD2, CD3, CD14, CD16, CD19, CD24, CD56, CD66b glycoprotein A, StemSep). The enriched pool of CD34+ or lineage depleted cells preferably comprises at least 40%, more preferably at least 60% and most preferably at least 80% cells of this type. In the case of the Isolex 300i, this comprises the following steps of the automated procedure (as per the Manufacturer's protocol and software version 2.5): 1. Cells are washed in DPBS supplemented with 0.41% sodium citrate and 1% of human serum albumin to remove platelets; 2. Cells are incubated with the anti-CD34 antibody (as supplied) for 15 minutes at room temperature and unbound antibody is removed by washing; 3. cells are transferred to the magnetic chamber for rosetting (30 minutes at room temperature); 4. magnet is applied to capture the CD34+ cell/magnetic bead complex and unbound cells are removed by washing; 5. bound cells are released by incubation with the release reagent PR34+; 6. cells are washed and transferred to the end product bag for subsequent processing.

Step 7-Washing Step #3 (Example 1; Day 5).

The cells are counted and washed by centrifugation or by using the Nexell CytoMate or similar and transferred into cell culture medium comprising Iscove's Modified Dulbecco's Medium (IMDM) supplemented with 10% heat inactivated Fetal Bovine Serum and, in a preferred embodiment with 50 ng/mL Stem Cell Factor (SCF) and 100 ng/mL Megakaryocyte Growth and Development Factor (MGDF). This is performed in IMDM supplemented with 10% heat inactivated Fetal Bovine Serum using a Nexell CytoMate washer program that washes cells from bag 4 through spinning membrane to wash bag 1 with the following parameters (Residual Fold Reduction=10; Maximum End Weight=20 mL; Source Bag Rinse=50 mL; or similar parameters). This is followed by transfer of cells from wash bag 1 to bag 3 end product, a LifeCell bag, with the following parameters (Tubing Rinse Volume=500 mL; Maximum Pump Rate=50 mL per minute; or similar parameters).

Step 8-Cell Culture (Example 1: Days 5-8).

The cells are placed at preferably 1×10⁵ to 5×10⁶ cells/ml into cell culture flasks, cell culture bags or in a preferred embodiment into 1,000 ml (390 cm²) Nexell Lifecell X-Fold Culture Bag (Nexell Therapeutics) or similar with Iscove's Modified Dulbecco's Medium plus 10% Fetal Bovine Serum (FBS) containing cytokines/growth factors. In a preferred embodiment this cytokine/growth factor mixture consists of Stem Cell Factor (50 ng/ml) and Megakaryocyte Growth and Development Factor 100 ng/ml). Steps 3-9 will result in up to 12×10⁷ HP cells or more (as assessed by CD34 positivity) per kg. Cell culture is conducted in a 37 degree Celsius humidified incubator with 5% CO₂ for 30-36 hours.

Step 9-Transduction Procedure (Example 1; Day 7).

The cells are harvested from the first flask or tissue culture bag, including in a preferred embodiment a Lifecell Culture Bag or similar and using the Cytomate device or similar, resuspended in retroviral supernatant such as a 200 microliter aliquot of a retroviral-containing medium, which in a preferred embodiment is produced from using AM-12 packaging cell line (Genetix Pharmaceuticals or ref Markowitz, D., Goff, S. & Bank, A. (1988). Construction and use of a safe and efficient amphotropic packaging cell line. Virology, 167, 400-406.)

In this example, the GMP grade retroviral supernatant was manufactured by BioReliance Corporation, Rockville, Md. under GMP conditions in a specialised facility. The ribozyme containing vector, RRz2, and methods for generating viral particles containing the ribozyme has been described in detail elsewhere (L-Q Sun, et al. (1998) “The design, production and validation of an anti-HIV type 1 ribozyme.” In Methods in Molecular Medicine. Vol 11. Therapeutic Applications of Ribozymes; pp 51-64, Humana Press). The vector producing cell line was plated at 3-4×10⁴ cells per cm² in 850 cm² roller bottles in DMEM supplemented with 10% heat inactivated fetal bovine serum, and cultured at 0.5-1.0 rpm in a humidified atmosphere in the presence of 5% CO2. When the cell density reached 9×10⁴ per cm², the culture medium was replaced with IMDM supplemented with 10% heat inactivated Fetal Bovine Serum and cultures for 4 hours. At time=4 hours, the culture supernatant was collected and stored at 2-8 degree Celsius, until all collections were prepared. The culture in IMDM supplemented with 10% heat inactivated Fetal Bovine Serum was repeated for an additional 5 hours and then again for a further 15 hours. In this way, 3 collections of virus containing medium were collected, pooled and 0.2 micron filtered prior to sterile filling 200 ml into 1 L Cryocyte bags (Nexell Therapeutics, Irving Calif.) and storage at −70 degrees Celsius.

The GMP grade virus supernatant was analysed to confirm absence of contamination by the following tests: Mycoplasma (1993 PTC), replication competent retrovirus co-cultivation assay, Isoenzyme analysis, in vitro adventitious virus assay, in vivo adventitious virus assay, sterility (membrane filtration) assay, bacteriostasis and fungistasis (membrane filtration) assay, general safety test, replication competent retrovirus amplification assay, residual DNA PCR assay, and bacterial endotoxin test (Limulus Amebocyte Lysate chromogenic assay). In addition the GMP material was tested for potency using a 3T3 infectivity assay. In the preferred embodiment, the titre of the retroviral supernatant is 1×10⁶ colony forming units per ml.

The 200 microliter aliquot was transferred into a second tissue culture container, one type of which is the Lifecell X-Fold Culture Bag which have a retrovirus transduction facilitating agent. Such agents include polybrene, protamine sulphate, cationic lipids or in a preferred embodiment, in a tissue culture container that has been pre-coated with RetroNectin (Takara Shuzo Co., Shiga, Japan) at 1-4 mcg/cm². The RetroNectin coating is conducted, for example, by addition of 0.8 mL of 1 mg/mL solution of RetroNectin to a 390 cm² LifeCell X-Fold Bag and incubation at 2-8 degrees Celsius for 16-48 hours. Any unbound RetroNectin is removed by washing twice with 60 mL DPBS. The Cytomate cell washing step is conducted using a program that washes cells with IMDM plus 10% heat inactivated Fetal Calf Serum from bag 4 through spinning membrane to wash bag 1 with the following parameters (Residual Fold Reduction=10; Maximum End Weight=20 mL; Source Bag Rinse=50 mL; or similar parameters). This is followed by transfer of cells in the retroviral supernatant (200 mL in a preferred embodiment) that has been thawed and supplemented with the cytokines SCF and MGDF at the concentrations as above, in a preferred embodiment. The transfer is from wash bag 1 to bag 3 end product which is the RetroNectin-coated LifeCell Bag with the following parameters (Tubing Rinse Volume=195 mL; Maximum Pump Rate=50 mL per minute; or similar parameters). The fluid path is then washed with an additional 50 mL of the IMDM plus 10% heat inactivated Fetal Calf Serum. The RetroNectin coated bag containing cells is placed in a 37 degree Celsius humidified incubator, with 5% CO₂. After 5-7 hours the transfer procedure is repeated using the CytoMate or similar; for this second transduction, cells are either transferred to a new tissue culture container (polybrene, protamine sulphate) or returned to the same or similar RetroNectin-coated container from which they came. The transfer with the Cytomate is identical to the procedure described for the first transduction. In a preferred embodiment, this is done in a fresh 200 mL aliquot of retroviral supernatant as above and cultured overnight. In other embodiments this repeat transduction is either not done or is repeated several times for similar periods of time. An aliquot of the retroviral supernatant(s) is collected for sterility testing. This growth/transduction procedure will result in up to 5×10⁷ gene-containing HP cells or more (as assessed by CD34 positivity) per kg of body weight. This number is determined by quantitative assay such as DzyNA PCR. The transduction efficiency will be at least 10% or greater, and preferably in the range from 30-50%, and more preferably greater than 50%.

Step 10-Harvest Cell Product (Example 1; Preferably on Day 8).

On the morning of day 8 (which in the preferred embodiment is day 3 of cell culture), cells are harvested and washed using standard cell centrifugation (1,500 rpm or 300 g, for 15 minutes or similar)or automated systems such as the Cytomate samples of cell culture. The Cytomate cell washing step is conducted using a program that washes cells with RPMI without phenol red plus 0.3% human serum albumin from bag 4 through spinning membrane to wash bag 1 with the following parameters (Residual Fold Reduction=1,000; Maximum End Weight=20 mL; Source Bag Rinse=50 mL; or similar parameters). This is followed by transfer of cells in RPMI plus 5% human serum albumin. The transfer is from wash bag 1 to bag 3 end product which is the transfer pack for the final infusion product with the following parameters (Tubing Rinse Volume=100 mL; Maximum Pump Rate=50 mL per minute; or similar parameters). This will yield up to 5×10⁷ gene-containing HP cells or more (as assessed by CD34 positivity) per kg in 100 mL RPMI plus 5% human serum albumin or similar carrier.

Step 11-Infusion Product (Example 1; Day 8).

Cells are thus resuspended in a physiologic infusion buffer containing 5% human serum albumin or similar as carrier. Aliquot samples are removed for sterility culture (aerobic bacteria, anaerobic bacteria, fungus, mycoplasma). Infusion product is not released until the results of endotoxin (LAL), hybridisation assay for Mycoplasma, bacterialgram stain testing, infusion cell viability and CD34+ cell purity are available. The dose of total CD34+ cells is calculated based on CD34+ cell number per kg body weight. Transduced cell number is determined after infusion, once the results of the DzyNA or similar PCR based assay is known.

Step 12-Infusion of Patient (Example 1; Day 8).

The CD34+ cell preparation is administered to the patient as appropriate. In a preferred embodiment, the patient receives a single infusion of 0.5-5×10⁷ transduced CD34+ cells or more per kilogram of body weight (cells/kg) in the physiologic infusion buffer containing 5% human serum albumin or similar as carrier. The dose of transduced CD34+ cells per patient will depend on the efficiency of each step of the mobilization, apheresis, isolation, culture and transduction procedures. The total number of CD34+ cells (transduced and non-transduced) is determined by cell counting and flow cytometry. The introduced gene-containing HP cells give rise to a chimeric hematopoietic system in which there is a percentage of gene-containing HP cells in the bone marrow. For the treatment of HIV/AIDS positive individuals, this percentage of gene-containing HP cells is at least 5%, preferably greater than 10% and more preferably greater than 20%.

### Example 2

### Use of DzyNA Technology to Detect and Quantify the Percentage Transduction of HP Cells and the Number of Gene Containing Progeny Cells

Step 1 Determination of the Percentage of Gene-Containing Cells in the Infusion Product by Use of Real-Time Quantitative PCR (DzyNA, see Citations Supra)

Step 2. Quantify the Number of Gene-Containing Progeny Cells Over Time Within the Individual by DzyNA Quantitative PCR (see Citations to Methodology Supra).

DzyNA-PCR is a general strategy for the detection of specific genetic sequences associated with disease or the presence of foreign agents. The method provides a system that allows homogeneous nucleic acid amplification coupled with real time fluorescent detection in a single closed vessel. The strategy involves in vitro amplification of genetic sequences using a DzyNA primer which harbors the complementary (antisense) sequence of a 10:23 DNAzyme. During amplification, amplicons are produced which contain active (sense) copies of DNAzymes that cleave a reporter substrate included in the reaction mix. The accumulation of amplicons during PCR is monitored by changes in fluorescence produced by separation of fluoro/quencher dye molecules incorporated into opposite sides of a DNAzyme cleavage site within the reporter substrate. Cleavage of this reporter substrate indicates successful amplification of the target nucleic acid sequence. Real time measurements can be performed on the ABI Prism 7700 Sequence Detection System or other thermocyclers that have the capacity to monitor fluorescence in real time (eg Corbett Rotor-Gene (Corbett Research, Sydney Australia), Stratagene Mx 4000 (Stratagene, LaJolla, Calif.) or Roche LightCycler (Roche, Germany).

DzyNA PCR protocols have been developed for analysis of vectors and therapeutic agents that contain the neomycin resistance gene. This assay has various uses including estimation of the percent transduction of cells and monitoring the presence and quantification of transduced cells, or their progeny, within patients undergoing gene therapy.

The reporter substrate, Sub G5-FD, was synthesised by Trilink Biotechnologies (California, USA). Sub G5-FD (illustrated below) is a chimeric molecule containing both RNA (shown below in lower case) and DNA nucleotides. It has a 3′ phosphate group that prevents its extension by DNA polymerase during PCR. Sub G5-FD was synthesised with FAM (F) and DABCYL (D) moieties attached to the “T” deoxyribonucleotides indicated. The cleavage of the reporter substrate can be monitored at 530 nm (FAM emission wavelength) with excitation at 485 nm (FAM excitation wavelength). SubG5-FD is shown here:

| | |
|---|---|
| 5′CACCAAAAGAGAAC(T-F)GCAATguT(T-D)CAG | (SEQ ID NO:5) |
| GACCCACAGGAGCG-p 3′ | |

Two PCR primers were synthesised by Sigma Genosys (NSW, Australia). The 5′ PCR primer (5L1A) hybridizes to the neomycin resistance gene. The 3′ primer (3L1Dz5) is a DzyNA PCR primer which contains (a) a 5′ region containing the catalytically inactive antisense sequence of an active DNAzyme and (b) a 3′ region which is complementary to the neomycin resistance gene. During PCR amplification using 5L1A and 3L1Dz5, the amplicons produced by extension of 5L1A contain both neomycin resistance sequences and catalytically active sense copies of a DNAzyme incorporated in their 3′ regions. The active DNAzyme is designed to cleave the RNA/DNA reporter substrate Sub G5-FD. The sequences of the PCR primers is shown here:

5L1A (5′ primer)

| | |
|---|---|
| 5′ GAG TTC TAC CGG CAG TGC AAA 3′ | (SEQ ID NO:6 |

3L1Dz5 (3′ DzyNA primer)

| | |
|---|---|
| 5′ CAC CAA AAG AGA ACT GCA ATT CGT | (SEQ ID NO:7) |
| TGT AGC TAG CCT TTC AGG ACC CAC AGG | |
| AGC GGC AAG CAA TTC GTT CTG TAT C 3′ | |

The human cell line CEMT4 was obtained from the American Type Culture Collection (Rockville, Md.). CEMT4 cells were transduced with retrovirus containing the neomycin resistance gene. Genomic DNA was isolated from CEM T4 cells, as well as CEMT4 cells transduced with retrovirus harboring the neomycin resistance gene, using the QIAGEN DNeasy Tissue Kit (QIAGEN Pty Ltd, Victoria, Australia. Cat #69504). DNA extracted from transduced cells was mixed with DNA from untransduced cells (by weight) to obtain the following percentage of transduced DNA −100%, 11%, 1.2%, 0.1%, 0.02% and 0% (ie 100% untransduced CEMT4).

Genomic DNA isolated from CEM T4 cells, as well as CEMT4 cells transduced with retrovirus harboring the neomycin resistance gene, was amplified by DzyNA PCR. Reactions contained 20 or 30 pmole 5L1A, 1 or 2 pmole 3L1Dz5, 10 pmol Sub G5-FD, 20U RNasin (Promega, Catalogue # N2515, Madison, Wis.), 20 pmol ROX passive reference dye and 1×QIAGEN HotStarTaq Master mix (QIAGEN Pty Ltd, Victoria, Australia. Catalogue # 203445) plus an additional 2.5 mM MgCl₂ in a total reaction volume of 40 μl. Duplicate reactions were set up which contained 1 μg of genomic DNA. Control reactions contained all reaction components with the exception of genomic DNA. The reactions were placed in an ABI Prism 7700 Sequence Detection System, denatured at 95° C. for 10 minutes, subjected to 10 cycles of 70° C. for 1 minute with a temperature decrease of 1° C. per cycle, and 94° C. for 1 minute. This was followed by a further 60 cycles at 60° C. for 1 minute and 94° C. for 30 seconds. Fluorescence was measured by the ABI Prism 7700 Sequence Detection System during the annealing/extension phase of the PCR.

Reactions with genomic DNA containing neomycin resistance gene showed an increase in FAM fluorescence at 530 nm over the fluorescence observed in control reactions. When 1 μg of genomic DNA containing DNA from transduced CEMT4 cells was analysed the calibration curve was linear over the range of 100 to 0.02% transduced cells (R² consistently >0.99). Reactions containing DNA from untransduced cells, or lacking DNA, did increase over the threshold level during 70 thermocycles of PCR. Calibration curves generated using standard amounts can be used to estimate the proportion of cells or DNA, containing the neomycin resistance gene, in an unknown sample. The experiments described in this example illustrate one set of reaction conditions that can be used to detect and quantify the neomycin resistance transgene. This protocol can be modified readily by those of ordinary skill in the art and used to detect the RNA transcript from the neomycin resistance gene following modification of the protocol and inclusion of reverse transcripts in the reaction mix.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Aguila, H. L., K. Akashi, J. Domen, K. L. Gandy, E. Lagasse, R. E. Mebius, S. J. Morrison, J. Shizuru, S. Strober, N. Uchida, et al. (1997). “From stem cells to lymphocytes: biology and transplantation.” Immunol Rev 157: 13-40.

Amado, R. G., R. T. Mitsuyasu, G Symonds, J. D. Rosenblatt, J. Zack, L.-Q. Sun, M. Miller, J. Ely, and W. Gerlach (1999) “A phase I trial of autologous CD34+ hematopoetic progenitor cells transduced with an anti-HIV ribozyme.” Human Gene Ther 10: 2255-2270.

Baum C, I. L. Weissman, A. S. Tsukamoto, A.-M. Buckle and B. Peault (1992) “Isolationof candidate human hematopoietic stem-cell population.” Proc Natl Acad Sci USA 89: 2804-2808.

Bender, M. A., Palmer, T. D., Gelinas, R. E. & Miller, A. D. (1987). Evidence that the packaging signal of Moloney murine leukemia virus extends into the gag region. J Virol, 61, 1639-1646.

Cooper, D., R. Penny, G. Symonds, A. Carr, W. Gerlach, L. Q. Sun and J. Ely (1999).

“A marker study of therapeutically transduced CD4+ peripheral blood lymphocytes in HIV discordant identical twins.” Hum Gene Ther 10(8): 1401-1421.

Haase, A. T., Henry, K., Zupancic, M., Sedgewick, G., Faust, R. A., Melroe, H., Cavert, W., Gebhard, K., Staskus, K., Zhang, Z.Q., Dailey, P. J., Balfour, H. H., Jr., Erice, A. & Perelson, A. S. (1996). Quantitative image analysis of HIV-1 infection in lymphoid tissue. Science, 274, 985-989.

Knop, A. E., A. J. Arndt, M. Raponi, M. P. Boyd, J. A. Ely and G. Symonds (1999).

“Artificial capillary culture: Expansion and retroviral transduction of CD4+ T lymphocytes for clinical application.” Gene Ther 6: 373-384.

Kohn, D. B., G. Bauer, C. R. Rice, J. C. Rothschild, D. A. Carbonaro, P. Valdez, Q. Hao, C. Zhou, I. Bahner, K. Kearns, et al. (1999). “A clinical trial of retroviral-mediated transfer of a rev-responsive element decoy gene into CD34(+) cells from the bone marrow of human immunodeficiency virus-1-infected children.” Blood 94(1): 368-371.

Levinsky, R. J. (1989). “Recent advances in bone marrow transplantation.” Clin Immunol Immunopathol 50(1 Pt 2): S124-132.

Marasco, W. A., S. Chen, J. H. Richardson, U. Ramstedt and S. D. Jones (1998).

“Intracellular antibodies against HIV-1 envelope protein for AIDS gene therapy.” Hum Gene Ther 9(11): 1627-1642.

McFarland, R. D., D. C. Douek, R. A. Koup and L. J. Picker (2000). “Identification of a human recent thymic emigrant phenotype.” Proc Natl Acad Sci U S A 97(8): 4215-4220.

Murray, J M, Kaufmann, G, Kelleher, A D, Cooper, D A. (1998). A model of primary HIV-1 infection. Mathematical Biosciences, 154:57-85

Murray, J (2001). HIV-1 RNA and DNA dynamics during treated and untreated primary HIV-1 infection Eighth Conference on Retroviruses and Opportunistic Infections Chicago.

Rossi, J. J., E. M. Cantin, N. Sarver and P. F. Chang (1991). “The potential use of catalytic RNAs in therapy of HIV infection and other diseases.” Pharmacol Ther 50(2): 245-254.

Santoro, S. W. and G. F. Joyce (1997). “A general purpose RNA-cleaving DNA enzyme.” Proc Natl Acad Sci USA 94(9): 4262-4266.

Sarver, N., E. M. Cantin, P. S. Chang, J. A. Zaia, P. A. Ladne, D. A. Stephens and J. J. Rossi (1990). “Ribozymes as potential anti-HIV-1 therapeutic agents.” Science 247: 1222-1225.

Schwartzberg, L. S., R. Birch, B. Hazelton, K. W. Tauer, P. Lee, Jr., R. Altemose, C. George, R. Blanco, F. Wittlin, J. Cohen, et al. (1992). “Peripheral blood stem cell mobilization by chemotherapy with and without recombinant human granulocyte colony-stimulating factor.” J Hematother 1(4): 317-327.

Sczakiel, G. and M. Pawlita (1991). “Inhibition of human immunodeficiency virus type 1 replication in human T cells stably expressing antisense RNA.” J Virol 65(1): 468-472.

Sempowski, G. D., Hale, L. P., Sundy, J. S., Massey, J. M., Koup, R. A., Douek, D. C., Patel, D. D. & Haynes, B. F. (2000). Leukemia inhibitory factor, oncostatin M, IL-6, and stem cell factor mRNA expression in human thymus increases with age and is associated with thymic atrophy. J Immunol, 164, 2180-7.

Smythe, J. A., D. Sun, M. Thomson, P. D. Markham, M. S. Reitz, R. C. Gallo and J. Lisziewicz (1994). “A Rev-inducible mutant gag gene stably transferred into T lymphocytes: An approach to gene therapy against human immunodeficiency virus type 1 infection.” Proc Natl Acad Sci USA 91(9): 3657-3661.

Sullenger, B. A., H. F. Gallardo, G. E. Ungers and E. Gilboa (1990). “Overexpression of TAR sequences renders cells resistant to human immunodeficiency virus replication.” Cell 63(3): 601-608.

Sun, L Q, D Warrilow, L Wang, C Witherington, J Macpherson and G Symonds (1994) “Ribozyme-mediated suppression of Moloney murine leukemia virus and human immunodeficiency virus type I replication in permissive cell lines.” Proc Natl Acad Sci USA 91: 9715-9719.

Sun, L. Q., W. L. Gerlach and G. Symonds (1996). The use of ribozymes to inhibit HIV replication. Catalytic RNA. F. Eckstein and D. Lilley. 10: 329-342.

Sun, L. Q., W. L. Gerlach and G. Symonds (1998). The design, production and validation of an anti-HIV typel ribozyme. Therapeutic Application of Ribozymes. K. J. Scanlon. Totowa, N.J., Humana Press Inc. 11: 51-64.

Sun, L. Q., J. Pyati, J. Smythe, L. Wang, J. Macpherson, W. Gerlach and G. Symonds (1995a). “Resistance to human immunodeficiency virus type 1 infection conferred by transduction of human peripheral blood lymphocytes with ribozyme, antisense or polymeric trans-activation response element constructs.” Proc Natl Acad Sci USA 92(16): 7272-7276.

Todd, A. V., C. J. Fuery, H. L. Impey, T. L. Applegate and M. A. Haughton (2000). “DzyNA-PCR: use of DNAzymes to detect and quantify nucleic acid sequences in a real-time fluorescent format.” Clin Chem 46(5): 625-630.

Tough, D. F. and J. Sprent (1995). “Life span of naive and memory T cells.” Stem Cells 13(3): 242-249.

Wang, L., C. Witherington, A. King, W. L. Gerlach, A. Carr, R. Penny, D. Cooper, G. Symonds and L. Q. Sun (1998). “Preclinical characterization of an anti-tat ribozyme for therapeutic application.” Hum Gene Ther 9(9): 1283-1291.

Zack, J. A., Arrigo, S. J., Weitsman, S. R., Go, A. S., Haislip, A. & Chen, I.S. (1990). HIV-1 entry into quiescent primary lymphocytes: molecular analysis reveals a labile, latent viral structure. Cell, 61, 213-22.

What is claimed is:

1. A method for introducing exogenous nucleic acid-containing Hematopoietic Progenitor (HP) cells into a subject comprising the steps of:
a) obtaining a cell sample comprising CD34+ HP cells from a subject;
b) concentrating the HP cells to provide a cell population comprising at least 40% HP cells;
c) introducing a vector comprising an exogenous nucleic acid product into the cell population wherein the exogenous nucleic acid product is capable of being expressed in said HP cells and wherein the cells are further cultured in vitro;
d) determining the number of such exogenous nucleic acid-containing HP cells such that upon delivery to a second or the same subject, the second or same subject receives a dose of at least 0.52×10⁶ gene containing CD34+ HP per kg body weight in a total cell population of 1.63×10⁶ CD34+ HP cells per kg body weight; and
e) introducing the dose to the subject.

2. The method of claim 1 wherein there are at least 10% exogenous nucleic acid containing HP cells in the bone marrow of the subject following bone marrow engraftment.

3. The method of claim 1 wherein if the number of exogenous nucleic acid containing cells is less than 0.52×10⁶ gene containing CD34+ HP per kg body weight, then the cells are frozen and the method of claim 1 is repeated or one or more mobilization and/or aphereses steps are added until the HP cell number is at least 0.52×10⁶ gene containing CD34+ HP per kg body weight.

4. A genetically modified CD34+ HP cell population produced by the method of claim 1.

5. The method of claim 1 wherein the number of exogenous nucleic acid containing CD34+ HP delivered to the second or same subject comprises at least 1×10⁷ cells/kg body weight.

6. The method of claim 1 wherein the number of exogenous nucleic acid containing CD34+ HP delivered to the second or same subject is at least 2×10⁷ cells/kg body weight.

7. The method of claim 1 wherein the number of exogenous nucleic acid containing CD34+ HP delivered to the second or same subject is at least 4×10⁷ cells/kg body weight.

8. The method of claim 1 wherein the number of exogenous nucleic acid containing CD34+ HP delivered to the second or same subject is at least 8×10⁷ cells/kg body weight.

9. The method of claim 1 wherein the number of exogenous nucleic acid containing CD34+ HP delivered to the second or same subject is at least 10×10⁷ cells/kg body weight.

10. The method of claim 1 in which the exogenous nucleic acid containing CD34+ HP cells produce exogenous nucleic acid containing progeny lymphoid and mycloid cells that can be detected in the individual's body for at least 1 year following the introducing step.

11. The method of claim 1 wherein the chimeric hematopoietic system produced as the result of the method of claim 1 comprises is at least 0.01% exogenous nucleic acid containing cells in any of the peripheral blood cell types within 4 years following the introducing step.

12. The method of claim 1 that produce a chimeric hematopoietic system that is at least 0.1% exogenous nucleic acid containing in any of the peripheral blood cell types within 4 years of treatment.

13. The method of claims 1 that produces a chimeric hematopoietic system that is at least 1% exogenous nucleic acid containing in any of the peripheral blood cell types within 4 years of treatment.

14. The method of claim 1 that produces a chimeric hematopoietic system that is at least 10% exogenous nucleic acid containing in any of the peripheral blood cell types within 4 years of treatment.

15. The method of claim 1 that produces a chimeric hematopoietic system that is at least 20% exogenous nucleic acid containing in any of the peripheral blood cell types within 4 years of treatment.

16. The method of claim 1 that produces a chimeric hematopoietic system that is at least 50% exogenous nucleic acid containing in any of the peripheral blood cell types within 4 years of treatment.

17. The method of claim 1 that produces a chimeric hematopoietic system that is at least 0.01% exogenous nucleic acid containing in a biopsy of bone marrow taken within 4 years of treatment.

18. The method of claim that produces a chimeric hematopoietic system that is at least 0.1% exogenous nucleic acid containing in a biopsy of bone marrow taken within 4 years of treatment.

19. The method of claim 1 that produces a chimeric hematopoietic system that is at least 1% exogenous nucleic acid containing in a biopsy of bone marrow taken within 4 years of treatment.

20. The method of claims 1 that produces a chimeric hematopoietic system that is at least 10% exogenous nucleic acid containing in a biopsy of bone marrow taken within 4 years of treatment.

21. The method of claims 1 that produces a chimeric hematopoietic system that is at least 20% exogenous nucleic acid containing in a biopsy of bone marrow taken within 4 years of treatment.

22. The method of claims 1 that produces a chimeric hematopoietic system that is at least 50% exogenous nucleic acid containing in a biopsy of bone marrow taken within 4 years of treatment.

23. The method of claim 1 wherein a myeloablation step is not performed on the patient.

24. The method of claim 1 wherein the method is used to introduce an anti-viral therapy to a patient.

25. The method of claim 1 wherein the anti-viral therapy is an anti-HIV therapy.

26. The method of claim 1 wherein the anti-HIV therapy is a ribozyme therapy.

27. The method of claim 1 wherein the ribozyme therapy includes the use of a RRz2 ribozyme.

28. The method of claim 26 wherein the subject is treated with a second anti-HIV therapy.

29. The method of claim 28 wherein the second anti-HIV therapy is a second ribozyme therapy.

30. The method of claim 28 wherein the second anti-HIV therapy is an antisense therapy, interfering RNA, RNA decoys, or intracellular antibodies.

31. The method of claim 1 wherein quantitative real time PCR is used to determine the percentage of CD34+ HP cells that contain the exogenous nucleic acid fragment or a transcription product of the exogenous nucleic acid.

32. The method of claim 1 wherein the PCR is used to determine the percentage of peripheral blood, lymphatic and bone marrow cells that contain the exogenous nucleic acid fragment or a transcription product of the exogenous nucleic acid.

33. The method of claim 32 wherein the exogenous nucleic acid is a ribozyme.

34. The method of claim 1 wherein the method is repeated until at least a bone marrow sample taken from the subject comprises at least 10% CD34+ HP cells.

35. A treatment regimen for an HIV infected subject comprising performing the method of claim 1 in combination with at least one other anti-HIV therapy.


SEQUENCE LISTING (7 sequences)

SEQ ID NO: 1 - RNA, 17 nt, artificial sequence
  target site for catalytic ribozyme RRz2
ggagccaguagauccua

SEQ ID NO: 2 - DNA, 36 bp, artificial sequence
  Rz2 ribozyme sequence
ttaggatcctgatgagtccgtgaggacgaaactggc

SEQ ID NO: 3 - DNA, 38 bp, artificial sequence
  Rz2 ribozyme sequence in vector
ttaggatcctgatgagtccgtgaggacgaaactggctc

SEQ ID NO: 4 - RNA, 38 nt, artificial sequence
  Rz2 ribozyme sequence
uuaggauccugaugaguccgugaggacgaaacuggcuc

SEQ ID NO: 5 - DNA, 41 bp, artificial sequence
  reporter substrate for  ribozyme
caccaaaagagaactgcaatgtttcaggacccacaggagcg

SEQ ID NO: 6 - DNA, 21 bp, artificial sequence
  PCR primer
gagttctaccggcagtgcaaa

SEQ ID NO: 7 - DNA, 76 bp, artificial sequence
  DzyNA primer
caccaaaagagaactgcaattcgttgtagctagcctttcaggacccacaggagcggcaagcaattcgttctgtatc